(19) United States
(12) Patent Application Publication
(10) Pub. No.: US 2004/0193174 A1
(43) Pub. Date: Sep. 30, 2004

(54) Spinal implant and driver tool

(76) Inventor: George W. Bagby, Spokane, WA (US)

Correspondence Address: WELLS ST. JOHN P.S., 601 W. FIRST AVENUE, SUITE 1300, SPOKANE, WA, 99201, US

(21) Appl. No.: 10/426,615

(22) Filed: Apr. 29, 2003

Related U.S. Application Data
(60) Provisional application No. 60/457,004, filed on Mar. 24, 2003.

(51) Int. Cl. A61B 17/58

(52) U.S. Cl. 606/99

(57) ABSTRACT

A tool for driving a bone joining implant is provided. The tool includes a drive body having a hollow tubular tool end with an inner bore at the leading end sized substantially the same as an inner bore of a bone joining implant to allow passage of contiguous bone peninsulas completely through the implant upon implantation. An implant is also provided.

### CROSS-REFERENCE TO RELATED APPLICATION

This application claims the benefit of U.S. Provisional Patent Application Serial No. 60/457,004, which was filed on Mar. 24, 2003, and which is incorporated by reference herein.

### TECHNICAL FIELD

The present invention pertains to surgical joining of bone bodies. More particularly, the present invention relates to instruments, implants, and methods for implant insertion and removal, and staged bone fusion or arthrodesis of bone bodies, such as spinal vertebrae.

### BACKGROUND OF THE INVENTION

Techniques are known for driving a foreign object between bone bodies to encourage fusion and arthrodesis between the bone bodies. For example, dowels of bone were driven via tapping between adjacent vertebrae according to one early technique for achieving arthrodesis, referred to as the well-known Cloward Technique for use in the human cervical spine. Threads were later added to the dowels of bone according to Otero-Vich German Patent Application Number 3,505,567, published Jun. 5, 1986. Subsequently, U.S. Pat. No. 4,501,269 to Bagby, issued Feb. 26, 1985, taught the use of a metal dowel in the form of a bone basket which is inserted between adjacent vertebrae. Even later, threads were added to such metal bone baskets, or cages.

In the cases of smooth bone dowels and bone baskets, or cages, the object is axially tapped into place using a driver tool. In the case of threaded bone dowels and threaded bone baskets, or cages, the object is torqued into place using a driver tool.

U.S. Pat. No. 6,447,545 B1 to Bagby, issued Sep. 10, 2002, discloses one tool and implant for driving a threaded implant between a pair of adjacent vertebrae having prepared bone beds. Such tool includes a pair of extendable and retractable drive pins that engage within drive holes provided in a rear open edge of the implant. However, such tool is relatively complicated, using threaded parts and apertures for the pins and retraction mechanism, and is prone to being contaminated with soft tissue and bodily fluids. Hence, post-operative cleaning is somewhat complicated and costly. Additionally, threaded portions are susceptible to being clogged with soft tissue. Furthermore, such a construction proves to be somewhat difficult to sterilize.

U.S. Pat. No. 6,371,986 B1 to Bagby, issued Apr. 16, 2002, discloses a spinal fusion device that receives peninsulas of contiguous, living bone from each of a pair of adjacent vertebral bodies that are received within a bore of the implant to enhance bone fusion and arthrodesis. The above-mentioned U.S. Pat. No. 6,447,545 B1 to Bagby also discloses another variation of such an implant and procedure. According to such implant constructions and procedures, the driver tool used to insert the implant extends within a rearmost inner bore portion of the implant, preventing the ability to insert the implant into a kerf between adjacent vertebral bodies such that the peninsulas of bone extend completely through the implant from a leading end to a trailing end so as to actually exit the trailing end of the implant, upon placement. The ability to provide contiguous, living peninsulas of bone that extend completely through and out the implant would be additionally desirable to further enhance bony fusion and arthrodesis between a pair of adjacent bone bodies.

Accordingly, improvements are needed to eliminate the above-described deficiencies.

### SUMMARY OF THE INVENTION

An instrument and implant are provided for driving a bone joining implant between a pair of bone bodies, such as inserting and removing a threaded vertebral body implant between a pair of adjacent vertebrae. The implant includes drive slots that mate with drive fingers on the instrument during insertion and removal of the implant while in threaded engagement with the adjacent vertebrae.

According to one aspect, a tool for driving a bone joining implant is provided. The tool includes a drive body having a hollow tubular tool end with an inner bore at the leading end sized substantially the same as an inner bore of a bone joining implant to allow passage of contiguous bone peninsulas completely through the implant upon implantation.

### BRIEF DESCRIPTION OF THE DRAWINGS

Preferred embodiments of the invention are described below with reference to the following accompanying drawings.

FIG. 1 is a perspective view of an implant system including an implant insertion tool usable for inserting and threading (as well as removing) a novel implant within the prepared bone beds of FIGS. 9-11.

FIG. 2 is a simplified sagittal view illustrating a hole saw and hole guide for cutting a cylindrical kerf within a pair of adjacent vertebrae for receiving the implant.

FIG. 3 is another perspective view of the implant system of FIG. 1 taken from a leading end adjacent the implant.

FIG. 4 is a perspective view taken from the driven end of the vertebral interbody implant of FIGS. 1 and 3.

FIG. 5 is a perspective view taken from the leading end of the vertebral interbody implant of FIGS. 1 and 3 for insertion within the prepared bone beds of FIG. 9.

FIG. 6 is a leading end view of the vertebral interbody implant of FIG. 4.

FIG. 7 is a side view of the vertebral interbody implant of FIG. 4.

FIG. 8 is a driven end view of the vertebral interbody implant of FIG. 4.

FIG. 9 is a simplified, sagittal and centerline view of the implant of FIGS. 4-8 prior to insertion.

FIG. 10 is a simplified, sagittal and centerline view of the implant of FIGS. 4-8 after insertion.

FIG. 11 is a surgical time simplified sagittal view of the implant of FIG. 10 received within the prepared bone beds of adjacent vertebrae and containing bone fragments immediately following implantation.

FIG. 12 is a leading end view of an alternative construction vertebral interbody implant.

FIG. 13 is a side view of the vertebral interbody implant of FIG. 12.

FIG. 14 is a driven end view of the vertebral interbody implant of FIG. 12.

FIG. 15 is a side view of a second alternative construction vertebral interbody implant.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

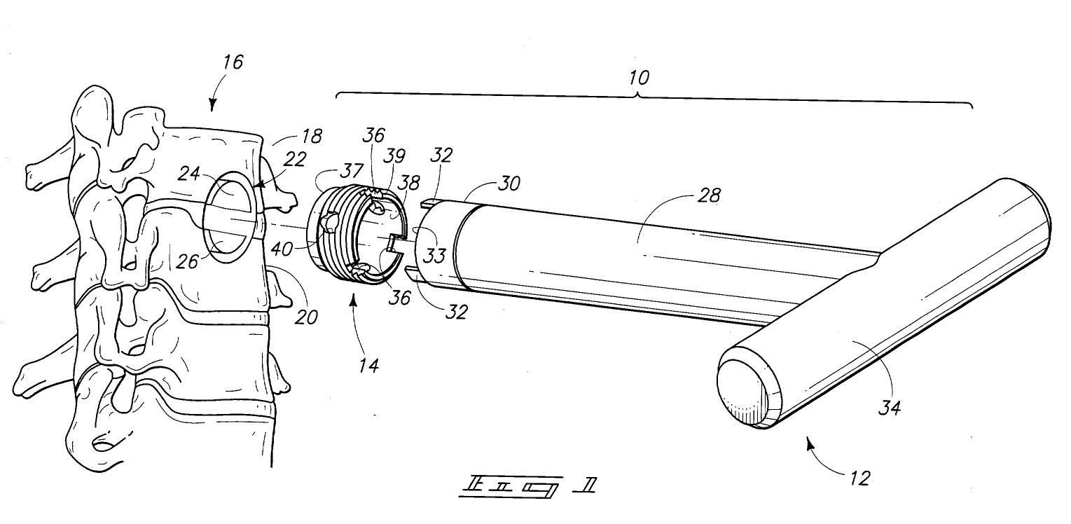

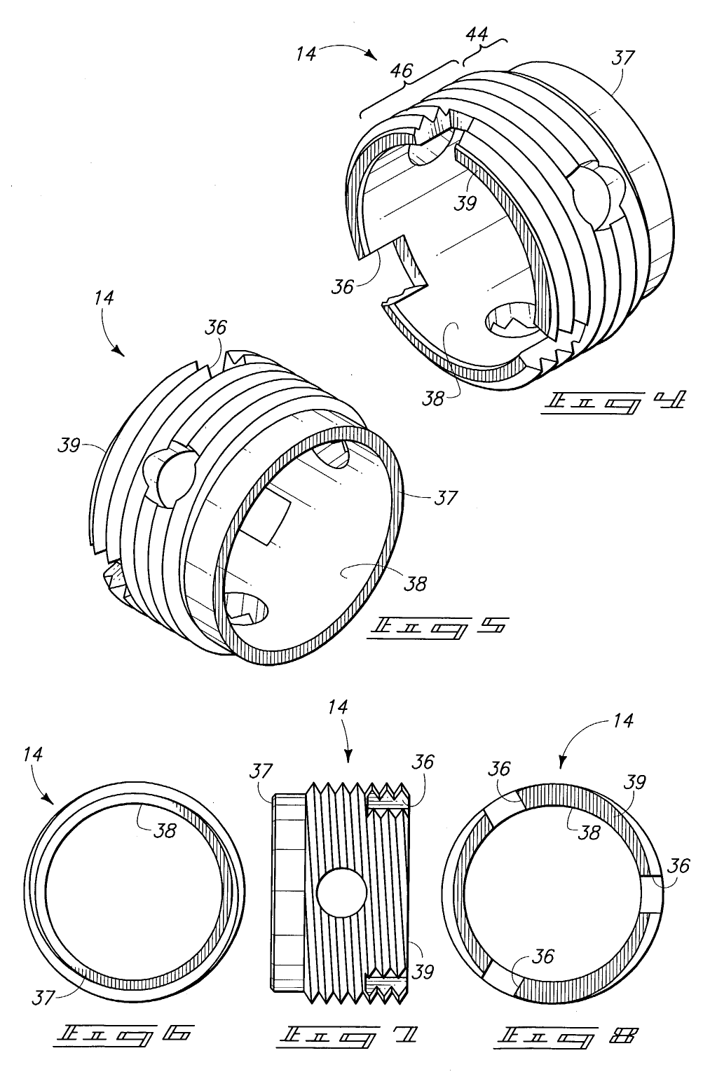

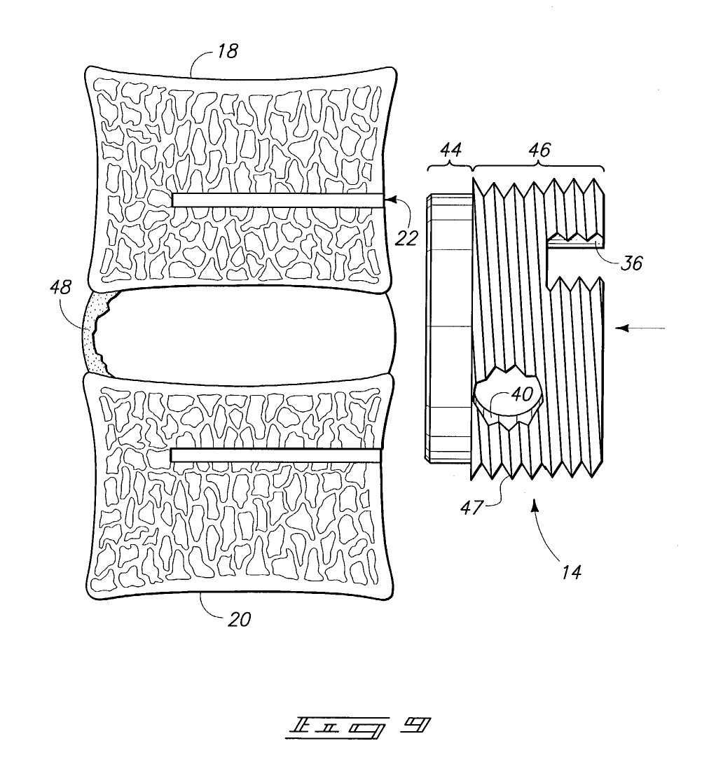

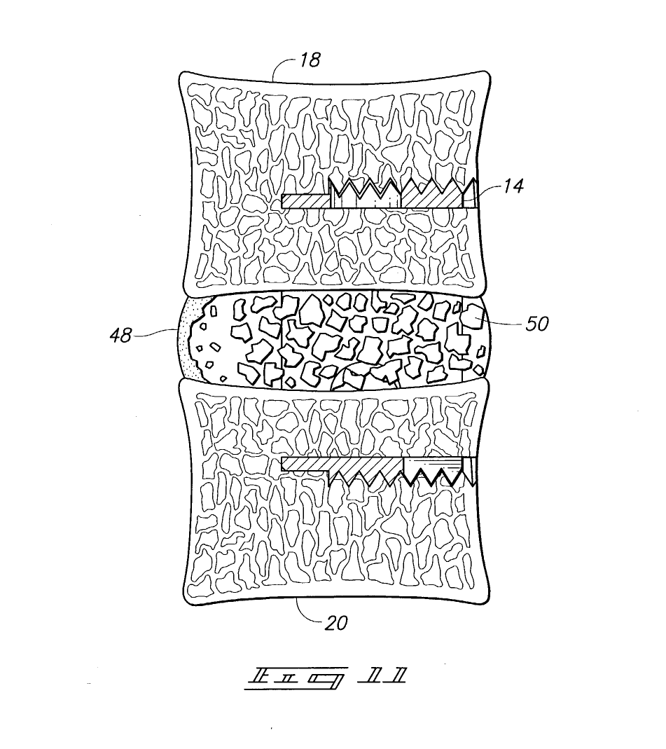

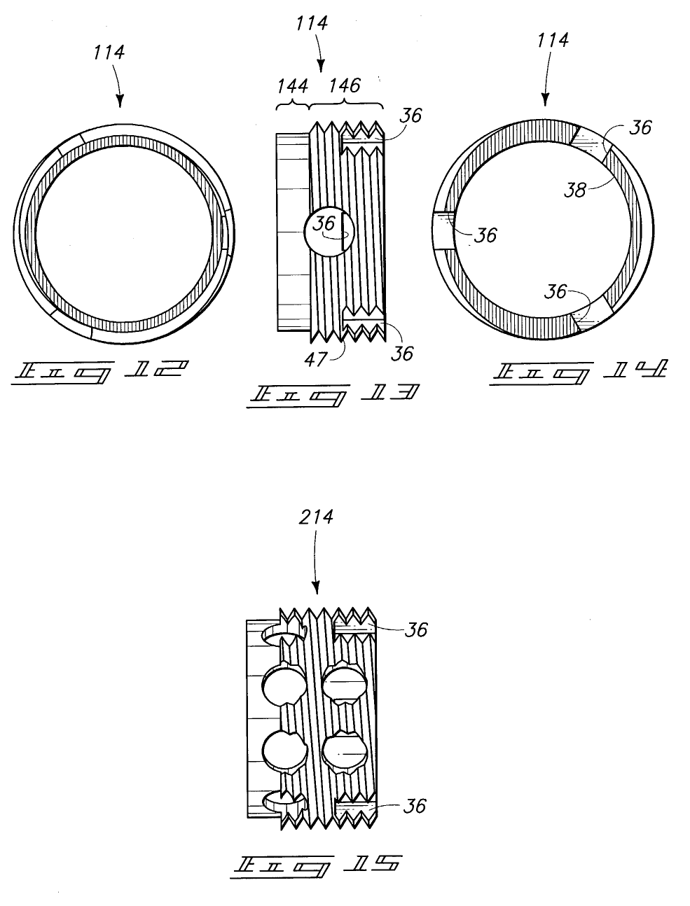

Reference will now be made to preferred embodiments of Applicant's invention. Three exemplary implementations are described below and depicted with reference to the drawings comprising various apparatus for inserting and removing bone joining devices such as spinal fusion devices between adjacent bone bodies. A first embodiment is shown and described below in a first configuration with reference generally to FIGS. 1-11. A second embodiment is shown and described below with reference to FIGS. 12-14. A third embodiment is shown and described below with reference to FIG. 15. While the invention is described by way of three preferred embodiments, it is understood that the description is not intended to limit the invention to these embodiments, but is intended to cover alternatives, equivalents, and modifications which may be broader than these embodiments such as are defined within the scope of the appended claims.

In an effort to prevent obscuring the invention at hand, only details germane to implementing the invention will be described in great detail, with presently understood peripheral details being incorporated by reference, as needed, as being presently understood in the art.

A preferred embodiment surgical implant system in accordance with the invention is first described with reference to FIGS. 1-11 and is identified by reference numeral 10. Such an implant system 10 is further described below with respect to an implant insertion instrument (or tool) 12 and an accompanying spinal fusion device (or implant) 14 that is configured to mate in inter-fitting engagement with instrument 12. Additionally, a first optional configuration is shown with reference to surgical implant system 110, as identified in FIGS. 12-14. Finally, a second optional configuration is shown with reference to surgical implant system 210, as identified in FIG. 15.

As shown in FIG. 1, surgical implant system 10 comprises implant insertion instrument 12 and spinal fusion device 14. Spinal fusion device 14 comprises a spinal fusion cage somewhat similar to that disclosed in U.S. Pat. No. 6,447,545 B1 to Bagby, issued Sep. 10, 2002, and incorporated herein by reference. However, spinal fusion device 14 includes the further additional features of special drive slots 36 that inter-engage with drive fingers 32 on instrument 12, and is suited for placement in a kerf 22 in a spine 16 of a human, between adjacent vertebrae 18 and 20. The specific details of how drive slots 36 interact with fingers 32 will be described below in greater detail. According to one construction, instrument 12 and implant 14 are constructed of metal, such as titanium or a surgical stainless steel.

As shown in FIG. 1, implant insertion instrument (or tool) 12 includes an axially extending and cylindrical drive body 28 having an open leading end portion 30 about which three equally spaced-apart engaging segments (or drive fingers) 32 extend axially forward from drive body 28. Instrument 12 also includes a T-shaped handle 34. Opening leading end 30 comprises a cylindrical wall portion having a thickness comparable to the tubular thickness of implant 14 and fingers 32 have a thickness and arcuate configuration that inserts in conforming relationship within each respective implant drive slot 36 of implant 14 so as to enable complete insertion of implant 14 so that bone projections (or peninsulas) 24 and 26 of vertebrae 18 and 20 extend completely through inner bore 38 of implant 14 beyond a trailing end 39 of implant 14 such that implant 14 is recessed within vertebral bodies 18 and 20 (see FIG. 11).

The ability to recess implant 14 such that bone projections 24 and 26 extend beyond the rearmost surface 39 of implant 14 enhances bone fusion and arthrodesis. Opening leading end 30 can extend partially along drive body 28, toward handle 34. Additionally, drive body 28 can be completely hollow. Further alternatively, drive body 28 can be solid and a bore is only provided in the vicinity of open leading end 30.

As shown in FIGS. 1 and 3, implant 14 is inserted using instrument 12 into a kerf 22 by axially displacing implant 14 via a leading end 37 within kerf 22. Subsequently, threads 47 (see FIG. 9) on implant 14 are engaged within kerf 22 by torquing implant 14 using T-shaped handle 34 on instrument 12 so as to drive the implant in clockwise rotation in threading engagement between bone bodies 18 and 20 until implant 14 is completely recessed within bone bodies 18 and 20 relative to the outer surfaces of such vertebral bodies 18 and 20, as well as relative to bone projections 24 and 26.

Although instrument 12 and implant 14 are shown with three fingers 32 and drive slots 36, respectively, it is understood that alternative constructions can use more fingers 32 and slots 36 such as four, five, six or more.

By using more than two fingers and slots, implant 14 is retained onto instrument 12 when instrument 12 is held with drive body 28 in a substantially horizontal configuration. The three fingers 32 and slots 36 cooperate to stabilize implant 14 onto instrument 12 in this configuration. Additionally, the rectangular configuration of fingers 32 and slots 36 provides added stability to implant 14 when mounted atop instrument 12. For example, implant 14 is prevented from rocking atop instrument 12.

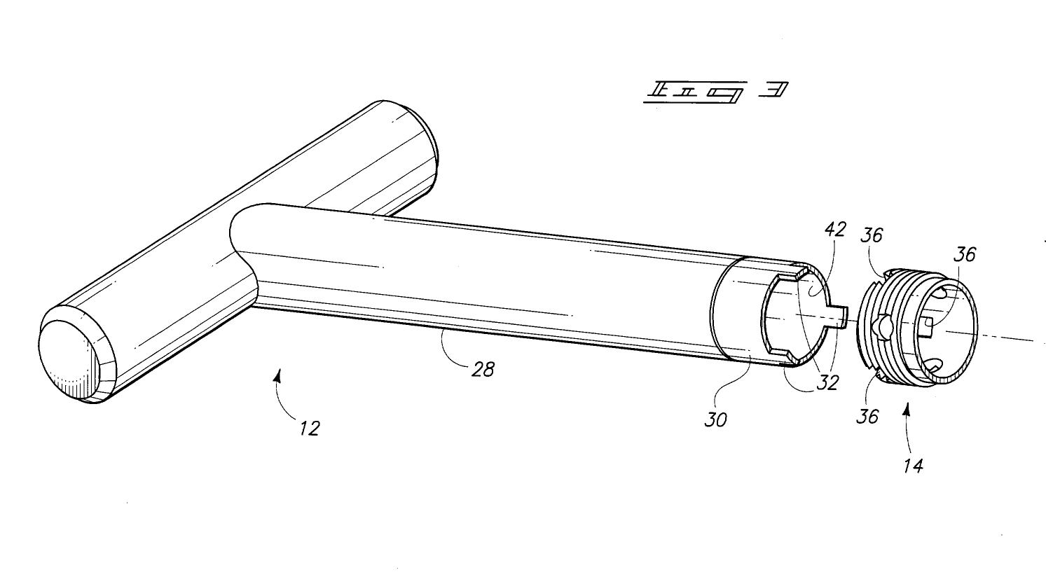

As shown in FIG. 4, a smooth insertion portion 44 is provided adjacent leading end 37 of implant 14, whereas an interlocking trailing portion 46 is provided aft of the smooth insertion portion 44, adjacent trailing end 39. In this manner, implant 14 is first axially inserted using the instrument, and subsequently the instrument and implant are torqued into position so as to thread the interlocking trailing portion into preferably existing threads that are formed within the kerf between the adjacent vertebral bodies. Further construction of implant 14 is shown illustrating the generally rectangular configuration of implant drive slots 36 by way of FIGS. 4-8.

FIG. 9 illustrates the positioning of implant 14 (omitting the instrument) prior to axially inserting smooth insertion portion 44 within kerf 22. Additionally, an intervertebral disk 44 is shown between vertebral bodies 18 and 20.

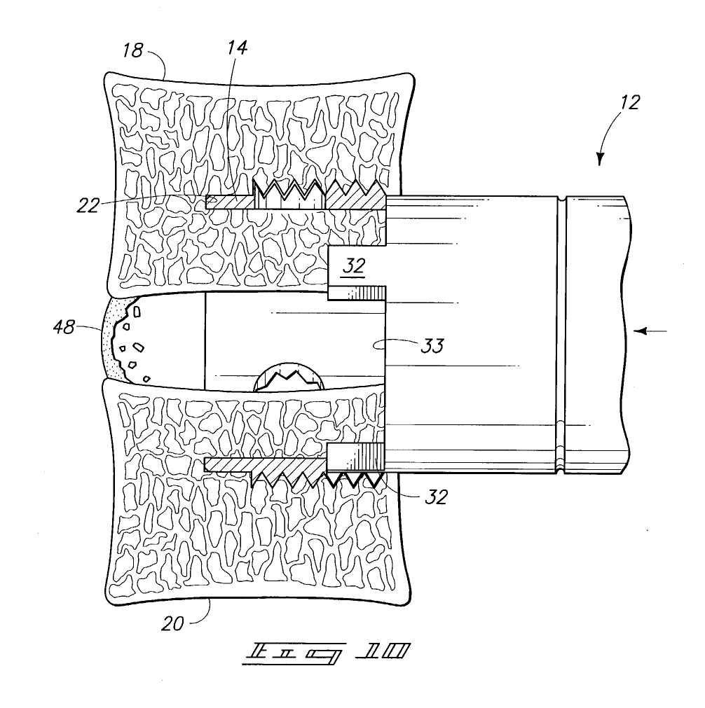

FIG. 10 illustrates the placement of implant 14 within kerf 22 after axially inserting the smooth insertion portion 44 and torquing in a clockwise direction interlocking trailing (or threaded) portion 46 between vertebrae 18 and 20.

As shown in FIG. 3, the hollow forming open leading end 30 of instrument 12 preferably extends rearward and within drive body 28 up to the point at which a circumferential outer scribe line is shown about drive body 28. Accordingly, sufficient axial penetration is provided for bone projections (or peninsulas) of vertebrae to extend completely through implant 14 and outward past the rearmost edge of implant 14 (and into open leading end 30) upon implantation. Subsequent to implantation, instrument 12 is removed from implant 14.

FIG. 11 illustrates the countersunk placement of implant 14 within vertebrae 18 and 20 and the provision of bone chips, or morsels, 150 that are packed inside and around the end of implant 14, following insertion. Such bone chips 150 facilitate earlier bone ingrowth and through-growth and eliminate the need to recover bone graft from a second surgical site, thereby enhancing bone fusion and arthrodesis (not shown) between adjacent vertebrae 18 and 20.

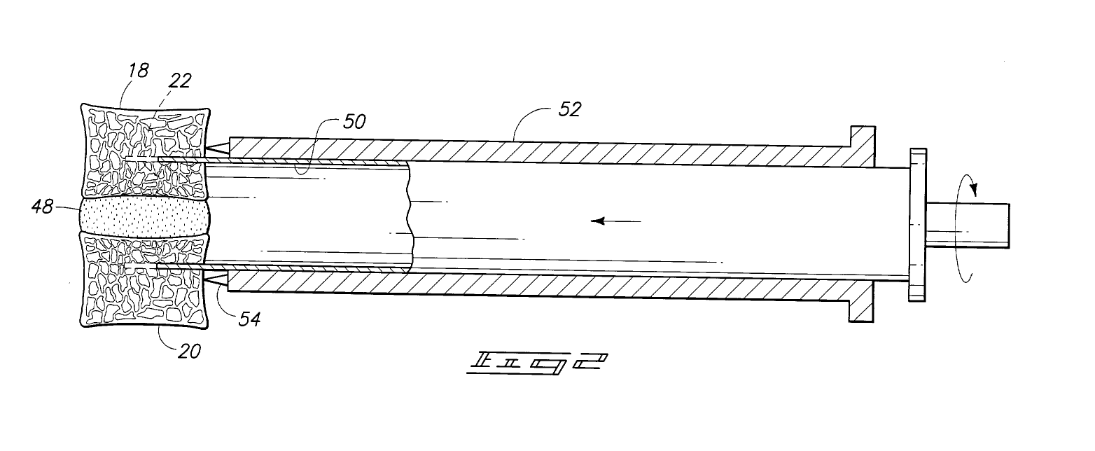

FIG. 2 illustrates the formation of kerf 22 using one or more hole saws 50 sized to cut a kerf of sufficient thickness to receive the implant. Hole saws 50 are driven in rotation by a drive tool (not shown) such as a hand torquing tool or a power drill. A tubular saw guide 52 is aligned and affixed to vertebrae 18 and 20 using a hammer and a plurality of set pins (or prongs) 54 which are partially driven into cortical outer bone of vertebrae 18 and 20. Subsequently, hole saw 20 is inserted within such tubular saw guide 52 and a kerf of sufficient depth is cut within vertebrae 18 and 20.

FIGS. 12-14 show a first alternative construction for an implant 114 having a smooth insertion portion 44 sized similar to that depicted for the implant 14 of FIGS. 4-8, but having a decreased thickness interlocking trailing portion 146.

Likewise, a second alternative construction implant 214 is illustrated in FIG. 15 having a similar smooth insertion portion and interlocking trailing portion, but including the addition of two rows of fenestrations provided through the circumferential wall of implant 214.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. A tool for driving a bone joining implant, comprising:
a drive body having a hollow tubular tool end with an inner bore at the leading end sized substantially the same as an inner bore of a bone joining implant to allow passage of contiguous bone peninsulas completely through the implant upon implantation.

2. The tool of claim 1 wherein the tubular tool end has an outer diameter that is substantially the same diameter as an outer diameter of an implant being inserted by the tool.

3. The tool of claim 1 wherein an outer diameter of the tubular tool end corresponds substantially with a minimum wall-thickness diameter of the implant.

4. The tool of claim 1 wherein the drive body has a threaded portion provided along an outer surface and an outer diameter of the tubular tool end corresponds substantially with a minor diameter of threads on the threaded portion.

5. The tool of claim 1 wherein the tubular tool end includes a plurality of axially extending engaging segments configured to interdigitate with complementary drive slots in a trailing end of an implant being inserted with the tool.

6. The tool of claim 5 wherein the engaging segments each comprise a generally rectangular finger having an arcuate cross-section conforming substantially with an arcuate cross-sectional surface of the bone joining implant.

7. The tool of claim 6 wherein at least three fingers are provided in equally spaced apart relation about the tubular tool end.

8. The tool of claim 7 wherein the tool end is cylindrical.

9. The tool of claim 1 wherein the hollow tubular tool end comprises a hollow cylindrical tool end provided at a proximal end of a cylindrical drive body, with a T-shaped handle provided at a distal end of the drive body.

10. A bone joining implant, comprising:
a cylindrical body having an outer surface including a threaded portion and an inner surface comprising a substantially uniform diameter through-bore extending completing through the body; and
at least three drive slots provided in a trailing end of the implant, each drive slot having a substantially rectangular configuration formed within an arcuate portion of the cylindrical body.

11. The bone joining implant of claim 10 wherein each drive slot is configured to receive a complementary rectangular drive finger that extends from a cylindrical end of a drive tool.

12. The bone joining implant of claim 10 wherein three drive slots are provided in cylindrically and equi-distant spaced apart relationship about a circumference on the trailing end of the cylindrical body.

13. The bone joining implant of claim 10 wherein at least one thread is provided on an outer surface of the cylindrical body adjacent a trailing end of the implant.

14. The bone joining implant of claim 13 wherein a smooth insertion portion is provided on the outer surface of the cylindrical body along a leading end of the implant.

15. The bone joining implant of claim 10 wherein the drive slots are configured to receive complementary, substantially rectangular drive fingers of a drive tool to facilitate rotational, threading engagement of the threaded portion of the cylindrical body within bone bodies being joined together.

16. The bone joining implant of claim 10 wherein the inner surface of the cylindrical body and the drive slots enable mating of the implant to a leading end of a drive tool with complementary drive fingers and a hollow leading end portion that substantially conform with a minimum wall thickness of a trailing end of the cylindrical body to facilitate insertion of the implant in a kerf of at least one bone body having a bone peninsula that extends completely through the implant so as to recess the implant relative to the at least one bone body upon implantation.

17. A tool and implant assembly, comprising;
a bone joining implant having a cylindrical body with at least one thread on an outer surface, an inner surface including a substantially uniform diameter through-bore extending completely through the body, and at least three drive slots provided in a trailing end of the implant, each drive slot of substantially rectangular configuration and arcuate in a direction transverse to the cylindrical body; and
a drive tool having drive fingers that mate with the drive slots for engaging and driving the implant including a drive body having a hollow tubular end with an inner bore at the leading end sized substantially the same as the inner bore of the implant to facilitate passage of contiguous bone peninsulas completely through the implant upon implantation within a kerf provided between a pair of bone bodies.

18. The tool and implant assembly of claim 17 wherein the tubular tool end has an outer diameter that is substantially the same diameter of a minor diameter of a thread on the implant.

19. The tool and implant assembly of claim 17 wherein the tool includes a cylindrical drive body having a T-shaped handle at a distal end and the hollow tubular end at a proximal end.

20. The tool and implant assembly of claim 17 wherein each drive slot is configured to receive a complementary one of the drive fingers to facilitate rotational threading of the implant into at least one bone body.
